(19) United States
(12) Patent Application Publication
(10) Pub. No.: US 2008/0255399 A1
(43) Pub. Date: Oct. 16, 2008

(54) Method for Production of Diphenylethylene

(76) Inventors: Alain Riondel, Forbach (FR); Christophe Navarro, Lahonce (FR); Jean-Philippe Gendarme, Saint-Avold (FR); Bernard Wechtler, Valmont (FR)

Correspondence Address: ARKEMA INC.;PATENT DEPARTMENT - 26TH FLOOR, 2000 MARKET STREET, PHILADELPHIA, PA, 19103-3222, US

(21) Appl. No.: 11/579,793

(22) PCT Filed: May 3, 2005
(86) PCT No.: PCT/FR05/01102

§ 371 Date: Nov. 20, 2007

(30) Foreign Application Priority Data
May 7, 2004 (FR) 0404943

(51) Int. Cl. C07C 7/05

(52) U.S. Cl. 585/430

(57) ABSTRACT

The invention relates to a method for production of diphenylethylene (DPE) comprising the steps of: (a) catalytic dehydrogenation of diphenylethane (DPA) in the presence of water, (b) addition of a light organic solvent to the mixture from step (a) and (c) decanting the mixture from step (b) with recovery of a flow comprising diphenylethylene as a mixture with the solvent added in step (b). The invention further relates to a given mixture of DPE, DPA and solvent and use thereof in polymerisation.

### TECHNICAL FIELD

A subject matter of the invention is a process for the preparation of diphenylethylene (DPE) by catalytic dehydrogenation of diphenylethane (DPA).

### STATE OF THE ART

Diphenylethylene (DPE) is used, for example, in the synthesis of triblock polymers, in particular SBM (styrene/butadiene/methyl methacrylate) polymers.

The synthesis of DPE by catalytic dehydrogenation in the presence of water starting from DPA is known, for example from SU472166. The document DE19814457 discloses the preparation of DPE from DPA by catalytic dehydrogenation. The purification of the DPE obtained is carried out on a packed distillation column in the presence of a third substance which is sodium hydroxide. The document DE19814459 discloses the synthesis of DPE from DPA in the presence of water over a catalyst, the reaction being carried out at the same time as the reaction for the dehydrogenation of ethylbenzene (EB) to give styrene. DPE is obtained as a mixture with DPA, EB and styrene. The DPE is again separated from the styrene by distillation or then used as is at the applicative level. In the conventional case of the synthesis by catalytic dehydrogenation starting from DPA in the presence of water, the crude DPE (mixture of DPE and DPA) is difficult to separate from the water by settling. This is because, in the mixture with DPA and water, separation by settling does not occur because of the absence of a significant difference in density between the products. Specifically, water exhibits a density of 1 while a 50/50 by weight DPA/DPE mixture exhibits a density of 1.0045.

The separation of the DPE in the above processes is not satisfactory with regard to the simplicity of the process.

The invention is thus targeted at improving the separation of DPE from a reaction mixture comprising DPE/DPA/water.

### SUMMARY OF THE INVENTION

The invention provides a process for the manufacture of diphenylethylene comprising the stages of: (a) catalytic dehydrogenation of diphenylethane in the presence of water; (b) addition of a light organic solvent to the mixture obtained in stage (a); and (c) separation by settling of the mixture obtained in stage (b) and recovery of a stream comprising diphenylethylene as a mixture with the solvent added in stage (b).

According to one embodiment, the light organic solvent is ethylbenzene.

According to one embodiment, the amount of solvent added represents, with respect to the combined weight of diphenylethylene and diphenylethane, from 10 to 50% by weight, preferably from 15 to 30% by weight.

According to one embodiment, the process additionally comprises, after the stage of separation by settling, a stage (d) of dehydration over a molecular sieve.

According to one embodiment, the stream comprising the diphenylethylene comprises, by weight with respect to the total weight of the stream:
40 to 80% of DPE;
1 to 30% of DPA;
5 to 50% of solvent;
preferably:
50 to 70% of DPE;
5 to 20% of DPA;
10 to 30% of solvent.

A further subject matter of the invention is this mixture and its use in polymerization, in particular in SBM polymerization.

### DETAILED ACCOUNT OF EMBODIMENTS OF THE INVENTION

The first stage of the process for the synthesis of DPE is a catalytic dehydration of DPA to DPE in the presence of water. This first stage is conventional and known in the art. The person skilled in the art knows the operating parameters to use during this reaction. Generally, this reaction is typically carried out under the following operating conditions:
temperature of: 400 to 700° C.;
pre-evaporation temperature: 270 to 400° C.;
pressure: typically of the order of one atmosphere or less;
HSV: 0.05 to 5 h⁻¹;
water/DPA ratio by weight of between 1:1 and 10:1.

The catalyst is any catalyst for the dehydrogenation of 1,1-diarylethane, in particular of DPA. Mention may be made of the catalysts of the Styromax® range. Mention may also be made of the following documents, which provide a general description of this catalytic dehydrogenation stage: SU472166, DE19814457, DE19814459, U.S. Pat. No. 4,365,103, U.S. Pat. No. 2,450,334, JP01013048, JP01013052, JP2215-734, EP0282066 and WO9949967.

A reaction mixture predominantly comprising DPE, DPA and water is obtained on exiting from this first stage. In this mixture, the DPE+DPA/water ratio corresponds substantially to the DPA/water starting ratio (taking into account the selectivity of the conversion of the DPA to DPE). Still in this mixture, the DPE/DPA ratio varies within the conventional limits.

A light organic solvent (density of less than 0.95, preferably of between 0.95 and 0.85) is added to this Mixture. Use is advantageously made of ethylbenzene EB (in what follows, the description is given with reference to this product but any other appropriate solvent can be substituted). The amount, with respect to the DPE+DPA sum, is generally between 10 and 50%, preferably 15 to 30%. A phase of separating by settling is subsequently carried out in a decanter for a residence time which can be between 120 and 10 minutes, preferably between 60 and 15 minutes. The decanter is any conventional decanter, for example of Grignard or centrifuge type, by coalescence, and the like. The temperature at which this separating by settling can be carried out can be between 50 and 20° C., preferably between 35 and 25° C.

Two phases are obtained, one aqueous and the other organic. The organic phase comprises the light solvent and the DPE and DPA dissolved in the latter. This organic phase stream comprises predominantly DPE in EB, with traces of water. These traces of water can be removed, for example by percolating through a molecular sieve. A person skilled in the art knows the molecular sieves suitable for the mixtures to be separated.

On exiting from the molecular sieve, a stream is obtained which comprises predominantly DPE with a small amount of DPA in EB and which comprises less than 100 ppm, preferably less than 50 ppm, of water. The relative proportions of DPE, DPA and EB in the solution can vary and depend on the performance of the catalytic dehydrogenation stage and on the water/DPA starting ratio, and on the amount of EB which is added to the dehydrogenate.

Such a mixture in a solvent, optionally a monomer solvent, can be used directly in SBM synthesis.

The invention is also targeted at such a specific mixture which comprises, by weight with respect to the total weight of the mixture:
40 to 80% of DPE, preferably 50 to 70%;
1 to 30% of DPA, preferably 5 to 20%;
5 to 50% of EB, preferably 10 to 30%;
optionally other components in an amount of less than 15%, preferably 10%.

Such a mixture can be used directly in synthesis, in particular in SBM synthesis. In comparison with a process for DPA/DPE and EB/S synthesis in parallel, as disclosed, for example, in DE19814459, the mixture comprises a larger part of DPE. This is because, in the document DE19814459, in table 2, for the most favorable case of a synthesis from a 50/50 EB/DPE mixture, the amount of DPE is only approximately 38% by weight of the final mixture.

### EXAMPLES

The following examples illustrate the invention without limiting it.

Use is made of a tubular reactor with a diameter of 45 mm which is heated to 570° C. under a pressure of 0.5 bar and which comprises 170 ml of Styromax® 3 catalyst. This reactor is fed with DPA and water, which are vaporized beforehand at 310° C., at respective flow rates of 52 and 156 g/h. On exiting from the reactor, after cooling the reaction mixture, ethylbenzene is added at the rate of 13 g/h and separation by settling is carried out at ambient temperature for 15 minutes. An organic phase is obtained which then comprises:
DPE: 61.2%;
DPA: 11.9%;
EB: 19.4%;
other products: 6.5%; and
water: 500 ppm.

After operating for 24 h, 1560 g of DPE are thus recovered and are subsequently treated over a molecular sieve (NK30 from Ceca) to remove the water. This dehydration is carried out by feeding, via the bottom, a column with a length of 1 m and a diameter of 24 mm containing 285 g of molecular sieve. The DPE flow rate is 456 g/h. After percolating through the sieve, the water content of the product is approximately 45 ppm.

What is claimed is:

1. A process for the manufacture of diphenylethylene comprising the stages of:
(a) catalytic dehydrogenation of diphenylethane in the presence of water;
(b) addition of a light organic solvent to the mixture obtained in stage (a); and
(c) separation by settling of the mixture obtained in stage (b) and recovery of a stream comprising diphenylethylene as a mixture with the solvent added in stage (b).

2. The process as claimed in claim 1, in which the light organic solvent is ethylbenzene.

3. The process as claimed in claim 1 or 2, in which the amount of solvent added represents, with respect to the combined weight of diphenylethylene and diphenylethane, from 10 to 50% by weight.

4. The process as claimed in claim 3, in which the amount of solvent added represents, with respect to the combined weight of diphenylethylene and diphenylethane, from 15 to 30% by weight.

5. The process as claimed in one of claims 1 to 4, additionally comprising, after the stage of separation by settling, a stage:
(d) of dehydration over a molecular sieve.

6. The process as claimed in one of claims 1 to 5, in which the stream comprising the diphenylethylene comprises, by weight with respect to the total weight of the stream:
40 to 80% of diphenylethylene;
1 to 30% of diphenylethane;
5 to 50% of solvent.

7. The process as claimed in claim 6, in which the stream comprising the diphenylethylene comprises, by weight with respect to the total weight of the stream:
50 to 70% of diphenylethylene;
5 to 20% of diphenylethane;
10 to 30% of solvent.

8. A mixture comprising, by weight with respect to the total weight of the mixture:
40 to 80% of diphenylethylene;
1 to 30% of diphenylethane;
5 to 50% of monomer solvent.

9. The mixture as claimed in claim 8, comprising, by weight with respect to the total weight of the mixture:
50 to 70% of diphenylethylene;
5 to 20% of diphenylethane;
10 to 30% of monomer solvent.

10. The use in polymerization of the mixture as claimed in claim 8 or 9 or of the mixture capable of being obtained by the process as claimed in any one of claims 2 to 7.

11. The use as claimed in claim 10, in which the polymerization is SBM polymerization.
